(19) United States
(12) Patent Application Publication
(10) Pub. No.: US 2026/0045340 A1
(43) Pub. Date: Feb. 12, 2026

(54) USER BEHAVIOR PROPOSAL DEVICE AND ESTIMATION MODEL GENERATION DEVICE

(71) Applicant: NTT DOCOMO, INC., Tokyo (JP)

(72) Inventors: Kengo MIEDA, Chiyoda-ku (JP); Takafumi YAMAUCHI, Chiyoda-ku (JP); Yuuki YAMADA, Chiyoda-ku (JP); Satoshi HIYAMA, Chiyoda-ku (JP)

(73) Assignee: NTT DOCOMO, INC., Tokyo (JP)

(21) Appl. No.: 18/854,760

(22) PCT Filed: Mar. 22, 2023
(86) PCT No.: PCT/JP2023/011251

§ 371 Date: Oct. 7, 2024

(30) Foreign Application Priority Data
May 11, 2022 (JP) 2022-078033

(51) Int. Cl. G16H 20/00

(52) U.S. Cl. CPC G16H 20/00

(57) ABSTRACT

A user behavior proposal device that can enable a user to carry out an executable behavioral change is provided. In the user behavior proposal device 100, an affinity calculating unit 102 calculates an affinity of one user with each life habits entry on the basis of a terminal log of the user. An information transmitting unit 105 transmits the calculated affinity to the user. The information transmitting unit 105 may notify one user of a behavior based on an affinity satisfying a predetermined condition as a recommended behavior as information based on the affinity.

### TECHNICAL FIELD

The present invention relates to a user behavior proposal device and an estimation model generation device that propose a behavior of a user.

### BACKGROUND ART

Patent Literature 1 discloses a life habits improvement support device that can promote a behavioral change of a target person such as a patient of a life habits-related disease or a candidate therefor. As described in Patent Literature 1, the life habits improvement support device includes an interest degree determining unit that determines a degree of interest of a target person in a plurality of healthy behaviors, a behavioral characteristics determining unit that determines behavioral characteristics of behaviors of the target person, and a provision unit that provides support information for supporting improvement of life behaviors of the target person to a target device of the target person on the basis of determination results from the interest degree determining unit and the behavioral characteristics determining unit.

### CITATION LIST

### Patent Literature

[Patent Literature 1] Japanese Unexamined Patent Publication No. 2021-26556

### SUMMARY OF INVENTION

### Technical Problem

In Patent Literature 1, in order to improve life habits through healthy behaviors of interest, support information for supporting improvement of life habits of a target person is provided to the target person, but the support information is not necessarily information which can be easily executed by the target person.

Therefore, in order to solve the aforementioned problem, an objective of the present invention is to provide a user behavior proposal device and an estimation model generation device that can enable a user to carry out a behavior change which can be easily executed by a user.

### Solution to Problem

A user behavior proposal device according to the present invention includes: an affinity calculating unit configured to calculate an affinity with each of a plurality of behaviors of one user on the basis of at least one of a terminal log of the one user, user attributes, and a questionnaire result; and an output unit configured to output a behavior with a high affinity as a recommended behavior to the one user.

### Advantageous Effects of Invention

According to the present invention, it is possible to propose an executable behavior to a user.

### BRIEF DESCRIPTION OF DRAWINGS

FIG. 1 is a block diagram illustrating a functional configuration of a user behavior proposal device 100 according to the present disclosure.

FIG. 2 is a diagram illustrating a system configuration of a data collection system including a data collection and analysis device 200 that collects a terminal log to train an affinity estimation model 100a.

FIG. 3 is a block diagram illustrating a functional configuration of the data collection and analysis device 200.

FIG. 4 is a diagram schematically illustrating details of machine learning using terminal log information.

FIG. 5 is a diagram schematically illustrating a gap of a user from a set target value.

FIG. 6 is a diagram illustrating a step count distribution of user A.

FIG. 7 is a flowchart illustrating operations of the user behavior proposal device 100 and the data collection and analysis device 200 according to the present disclosure.

FIG. 8 is a flowchart illustrating operations of the data collection and analysis device 200.

FIG. 9 is a diagram schematically illustrating a result including a degree of risk contribution and an affinity.

FIG. 10 is a diagram illustrating an example of a hardware configuration of the user behavior proposal device 100 and the data collection and analysis device 200 according to one embodiment of the present disclosure.

### DESCRIPTION OF EMBODIMENTS

Hereinafter, an embodiment of the present invention will be described with reference to the accompanying drawings. As much as possible, the same elements will be referred to by the same reference signs and repeated description thereof will be omitted.

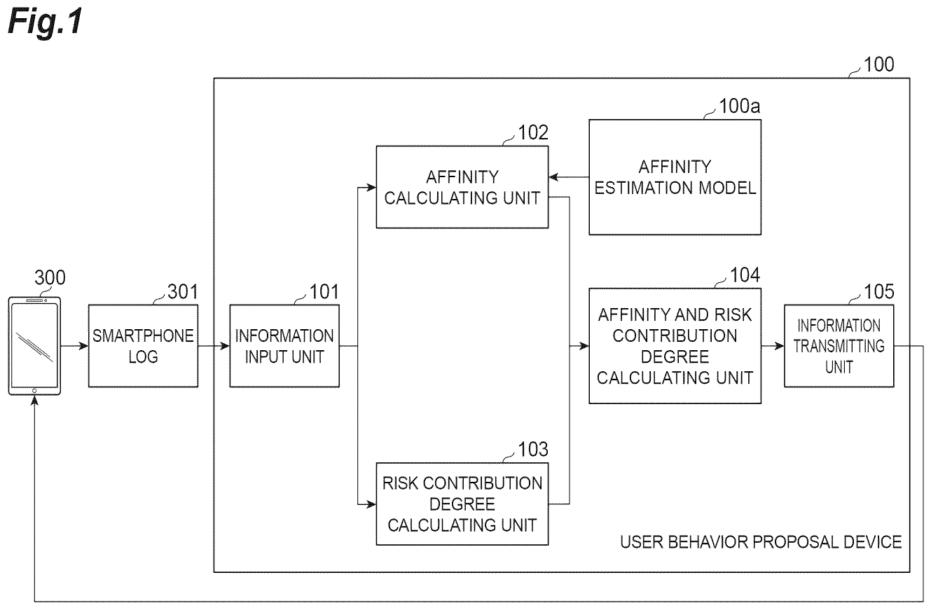

FIG. 1 is a block diagram illustrating a functional configuration of a user behavior proposal device 100 according to the present disclosure. The user behavior proposal device 100 is a device that proposes a user behavior corresponding to an affinity and a degree of risk contribution on the basis of a terminal log of a terminal 300. In the present disclosure, an affinity represents an acceptability of a user for a life habits entry indicating a user behavior. The degree of risk contribution represents a magnitude of an influence of a life habits entry indicating a user behavior on a health condition of a user. For example, the arbitrary health condition may be a risk of a life habits-related disease in the current or future or a current infection risk. A higher affinity means a behavior (life habits entry) which is more acceptable to a user. A higher degree of contribution means a behavior more contributing to improvement or change in health. In the present disclosure, the user behavior proposal device 100 proposes a user behavior corresponding to at least the affinity.

The user behavior proposal device 100 includes an information input unit 101, an affinity calculating unit 102, a risk contribution degree calculating unit 103, an affinity and risk contribution degree calculating unit 104, an information transmitting unit 105, and an affinity estimation model 100a.

The information input unit 101 is a part that receives an input of a smartphone log 301 which is a terminal log of the terminal 300. The smartphone log 301 may be an average value in a predetermined period or may be information acquired at any timing.

The affinity calculating unit 102 is a part that inputs the smartphone log 301 to the affinity estimation model 100a to calculate a user behavior and an affinity thereof. The affinity calculating unit 102 may input user attributes or a questionnaire answer result to the affinity estimation model 100a instead of or in addition to the smartphone log 301. The user attributes are information such as gender, age, residence, employment, and work type of a user. The user attributes may be stored in the terminal 300, or contractor information of a mobile phone may be used. The questionnaire answer result includes, for example, information such as a behavior which a user wants to adopt, an executable behavior, a hobby, tastes, a character, or a goal and is information which the user has input using the terminal 300.

The risk contribution degree calculating unit 103 is a part that calculates a degree of contribution to a health risk of a user with respect to a user's behavior and specifically calculates the degree of contribution using SHAP (XAI). SHAP is a technique of analyzing a machine learning model and is a technique for indicating how an entry input to an estimation model contributes to estimation of an arbitrary health condition. In addition to SHAP, LIME, Deep LIFT, or the like is known, and these may be used.

The affinity and risk contribution degree calculating unit 104 is a part that calculates a score indicating a degree of recommendation of a behavior based on an affinity and a degree of risk contribution for each user behavior. In the present disclosure, the score is calculated by multiplying an affinity by a degree of risk contribution for each user behavior, but is not limited thereto and the score may be calculated using another method. For example, addition may be used instead of multiplication, or multiplication or addition may be performed after an arbitrary weight is added to one of the affinity and the degree of risk contribution.

The information transmitting unit 105 is a part that transmits a score for each user behavior to the terminal 300.

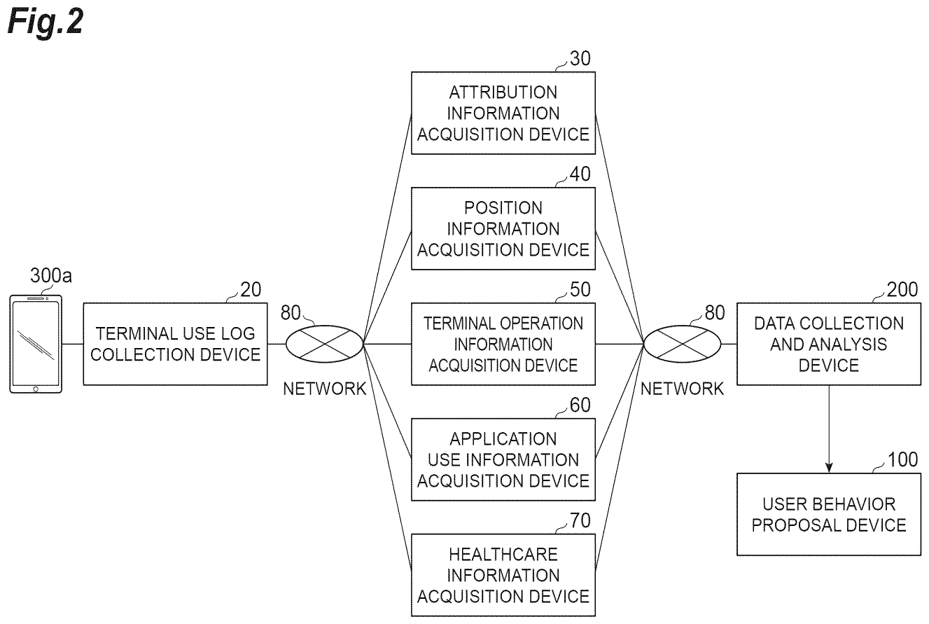

An affinity estimation model 100a which is used by the affinity calculating unit 102 will be described below. FIG. 2 is a diagram illustrating a system configuration of a data collection system including a data collection and analysis device 200 that collects a terminal log to train an affinity estimation model 100a.

As illustrated in FIG. 2, the data collection system includes a terminal use log collection device 20, an attribute information acquisition device 30, a position information acquisition device 40, a terminal operation information acquisition device 50, an application use information acquisition device 60, a healthcare information acquisition device 70, other acquisition devices, and a data collection and analysis device 200.

In FIG. 2, the terminal use log collection device 20 collects terminal log information of the terminal 300. The terminal log information is a use log of the terminal 300, and examples thereof include attribute information, position information, terminal operation information, application use information, and other healthcare information. These types of information are information which is acquired by various sensors provided in the terminal 300. The terminal 300 measures a position thereof using GPS or the like and can acquire a user behavior and health conditions using a gyro sensor or the like. The terminal use log collection device 20 collects the terminal log information acquired in the terminal 300.

The attribute information acquisition device 30 is a device that acquires attribution information in the terminal 300 (attribute information of a user) from the terminal use log collection device 20. The position information acquisition device 40 is a device that acquires position information of the terminal 300 from the terminal use log collection device 20. The terminal operation information acquisition device 50 is a device that acquires terminal operation information of the terminal 300 from the terminal use log collection device 20. The application use information acquisition device 60 is a device that acquires application use information of the terminal 300 from the terminal use log collection device 20. The healthcare information acquisition device 70 is a device that acquires healthcare information of the user of the terminal 300 from the terminal use log collection device 20. In addition, a device that acquires a questionnaire answer result from the user may be provided.

The data collection and analysis device 200 acquires at least one of various types of terminal log information, attribute information and a questionnaire answer result from the devices and generates an affinity estimation model 100a. The user behavior proposal device 100 acquires the generated affinity estimation model 100a and proposes a behavior with an affinity with the user.

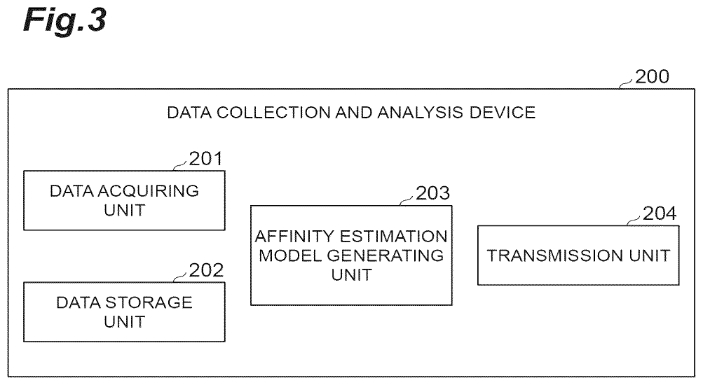

FIG. 3 is a block diagram illustrating a functional configuration of the data collection and analysis device 200. As illustrated in the drawing, the data collection and analysis device 200 includes a data acquiring unit 201, a data storage unit 202, an affinity estimation model generating unit 203, and a transmission unit 204.

The data acquiring unit 201 is a part that acquires at least one (hereinafter simply referred to as terminal log information) of various types of terminal log information, attribute information and a questionnaire answer result from the acquisition devices and acquires improvement data from the terminal log information. The improvement data is data indicating whether a life habits entry has been improved between before and after a behavioral change message as will be described later. The behavioral change message may be sent by the data collection and analysis device 200 or a may be sent by another message transmission server (not illustrated). A date and time at which the behavioral change message is transmitted is stored in the data storage unit 202.

The data storage unit 202 is apart that stores the acquired terminal log information and the acquired improvement data. The data storage unit 202 also stores reference information including a target behavior of the behavioral change message and a transmission date and time thereof. The data acquiring unit 201 acquires the improvement data using the reference information at the time of acquisition of the improvement data.

The affinity estimation model generating unit 203 is a part that generates an affinity estimation model 100a for a life habits entry from the terminal log information and the improvement data. The affinity estimation model generating unit 203 may generate the affinity estimation model 100a using the user attributes or the questionnaire answer result in addition to or instead of the terminal log information. Similarly, the improvement data may be acquired from the questionnaire answer result.

The transmission unit 204 is a part that transmits the generated affinity estimation model 100a to the user behavior proposal device 100.

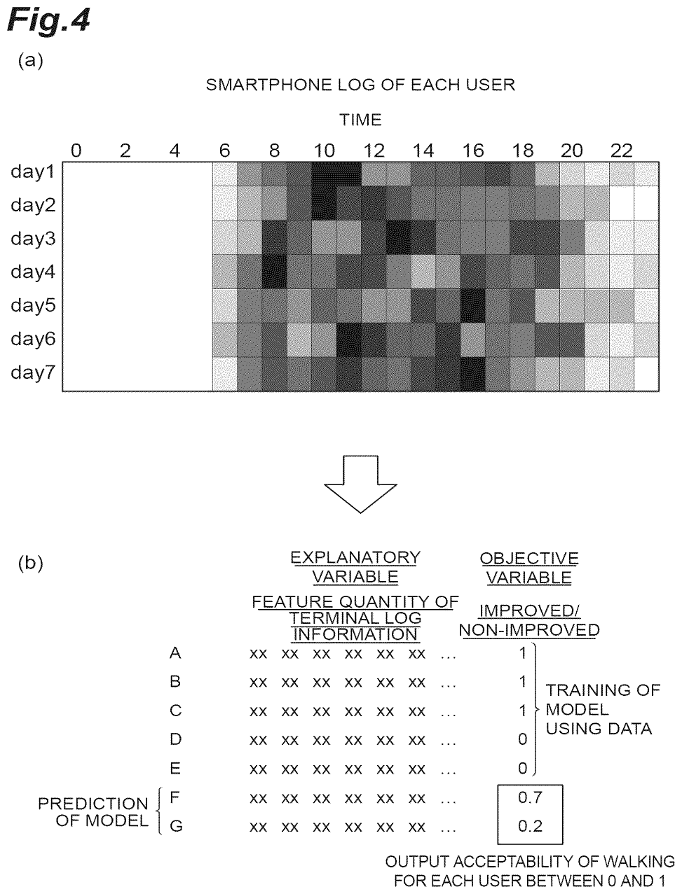

A detailed process of the affinity estimation model generating unit 203 will be described below. FIG. 4 is a diagram schematically illustrating details of machine learning using terminal log information. FIG. 4(a) illustrates a heat map based on the terminal log information. In FIG. 4, the terminal log information indicates a step count. Walking is a life habits entry and is a user behavior for improving health of each user. Other terminal log information may be used.

In FIG. 4(a), the horizontal axis represents a time period, and the vertical axis represents a day in one week (or a week day). In the drawing, the step count is expressed by shading intensities. For example, a light part indicates 100 steps, and a dark part indicates 1000 steps.

FIG. 4(a) illustrates a heat map of a user with respect to a step count, but the present disclosure is not limited thereto. Other terminal log information, other life habits entries, and a plurality of heat maps of other users are used in the present disclosure.

FIG. 4(b) illustrates a feature quantity of terminal log information and improvement data in a predetermined period using the heat maps. In FIG. 4(b), a record is generated for each user, and terminal log information converted to a feature quantity on the basis of a plurality of heat mas (information such as a step count, bedtime, and an amount of food) is illustrated as an explanatory variable. For example, for use as an explanatory variable, the step count, the bedtime, and the like in the terminal log information are converted to a feature quantity by calculating an average value in a predetermined period. The predetermined period is, for example, an average value in two weeks tracing back to the future from a reference time. The reference time indicates a time point at which a behavioral change message is transmitted as will be described later. In FIG. 4(b), feature quantities such as a step count, the position information, and a smartphone use situation in the predetermined period before the reference time are illustrated as explanatory variables. The predetermined period is not limited to an average value in two weeks and may be an average value in one week. The predetermined period is not limited to the average value, and other statistical information such as a sum value may be used.

In FIG. 4, terminal log information is converted to a feature quantity using a heat map, but the present disclosure is not limited thereto. The heat map is a means for visualizing a user's behavior, and the heat map is essential for preparing a feature quantity.

User attributes and a questionnaire answer result in addition to the terminal log information may be used to generate the affinity estimation model 100a. The user attributes are information such as gender, age, residence, employment, and work type of a user as described above. The questionnaire answer result includes, for example, information such as a behavior which a user wants to adopt, an executable behavior, a hobby, tastes, a character, or a goal and is information which a user has input using the terminal 300. These types of information may be converted to feature quantities.

Regarding improvement data, whether there is improvement may be determined on the basis of change of user attributes or change of a questionnaire answer result.

Accordingly, the affinity estimation model 100a may be trained, for example, by performing machine learning using user attributes (such as age and sex) as explanatory variables and using improvement data based on the questionnaire answer result as an objective variable.

A behavioral change message for changing a behavior is sent to each user (the terminal 300a) in advance to generate the affinity estimation model 100a. The objective variable is information based on whether each user has changed a behavior between before and after the behavioral change message has been sent. In FIG. 4(b), 1 is recorded when a user's life habits entry has been improved in response to the behavioral change message, and 0 is recorded when it has not been improved, which are handled as the objective variable.

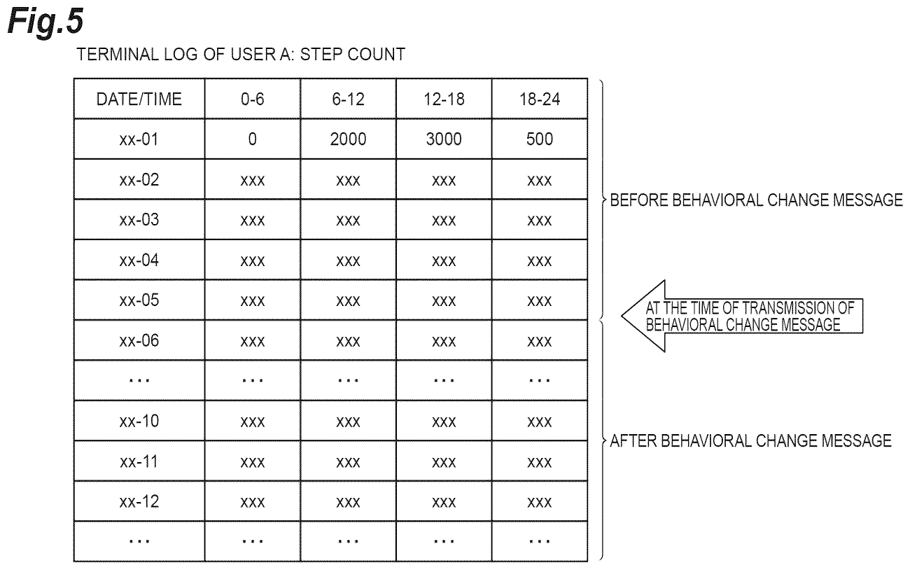

FIG. 5 is a diagram schematically illustrating terminal log information of a step count of user A. A step count every 6 hours is illustrated for the purpose of convenience of explanation, but the present disclosure is not limited thereto. As illustrated in the drawing, the step count is 0 between 00:00 and 06:00 on a first date of xx month, and the step count is 2000 between 06:00 to 12:00. In FIG. 5, a behavioral change message is transmitted between a fifth date of xx month and a sixth date of xx month. The behavioral change message includes, for example, details such as “let's walk for health.”

The objective variable in FIG. 4 is set to 1 when the step count has been improved in predetermined periods (for example, two weeks) before and after the behavioral change message has been sent and set to 0 when the step count has not been improved. Regarding improvement of the step count, an average value of the step count in the predetermined period (for example, two weeks) before the behavioral change message has been sent and an average value in the predetermined period (for example, two weeks) after the behavioral change message has been set are compared, and it can be determined that the step count has been improved when the average value of the step count after the behavioral change message is larger. For example, a threshold value which is larger by 10% or more than the average value may be used. The predetermined periods before and after the behavioral change message has been sent are preferably the same period.

When a change of the bedtime such as “go to bed early” is required using the behavioral change message, whether the bedtime has been improved based on a terminal log is used as the objective variable. Improvement in this case indicates that the bedtime become sooner. For example, it is assumed that the bedtime becomes sooner by 30 minutes.

The improvement data may be acquired from a questionnaire answer, or a questionnaire may be transmitted to a user before and after the reference time and whether the step count has been improved may be determined from a questionnaire result thereof. For example, when a step count is inquired of a user using a questionnaire and the step count is increased in a questionnaire after the reference time, it can be determined that the step count has been improved. Whether the step count has been improved may be determined using a qualitative question for ascertaining an intention change of a user such as whether an intention to increase the step count has been improved instead of a quantitative question for ascertaining whether the step count has been increased.

The affinity estimation model 100a is generated on the basis of the explanatory variables and the objective variables. In FIG. 4, a heat map of the step count is used, and this is an example. In addition, application use information, terminal operation information, healthcare information, and the like can be used as terminal log information, and the affinity estimation model 100a for determining an affinity of a step count, the bedtime, the wake-up time, hours of sleep, or the like which is a life habits entry can be generated using the information.

A target value may be included in a message for the behavioral change for the purpose of improvement in health. This is for visualizing an affinity between a user and a life habits entry to be changed. In the present disclosure, by setting an average value±1σ for each user, it is possible to realize setting of a target value without unevenness in difficulty in a behavioral change between users.

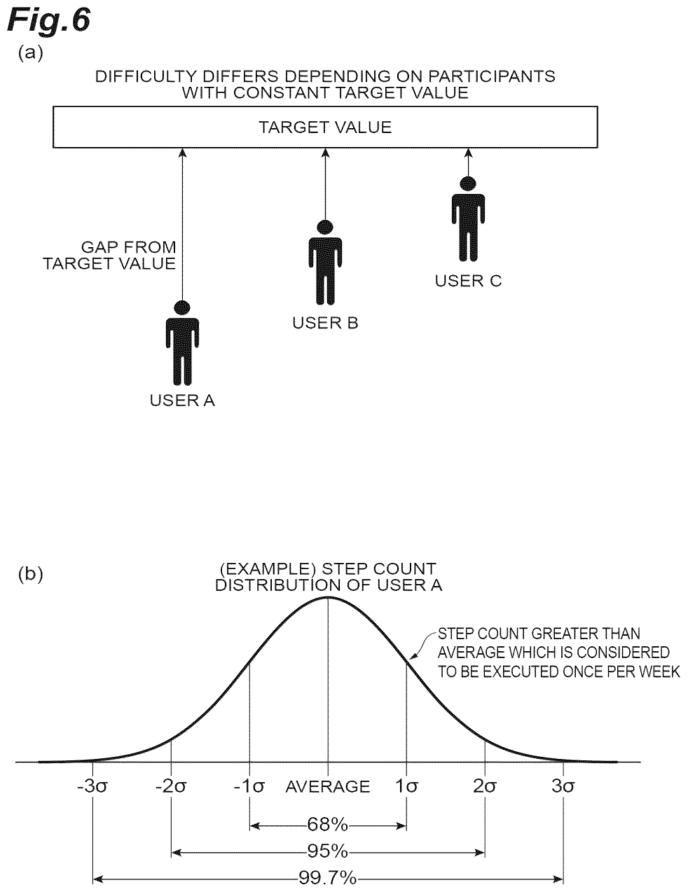

FIG. 6 is a diagram schematically illustrating a gap of a user from a set target value. When the target value is uniformly set as illustrated in FIG. 6(a), the difficulty of achievement of the target value may increase according to users. It is assumed that user A does not walk much in the normal state. However, in a relationship with other user B and user C, user A may feel a gap from the target value when a high target value is set.

FIG. 6(b) is a diagram illustrating a step count distribution of user A, and it is preferable that a step count slightly larger than the average value be set as the target value. In this way, it is preferable to set the target value on the basis of the step count distribution (the average value) measured for each user. In FIG. 6(b), the target value is set on the basis of to using a variance o. The present disclosure is not limited thereto, and ±2σ or ±3σ may be used.

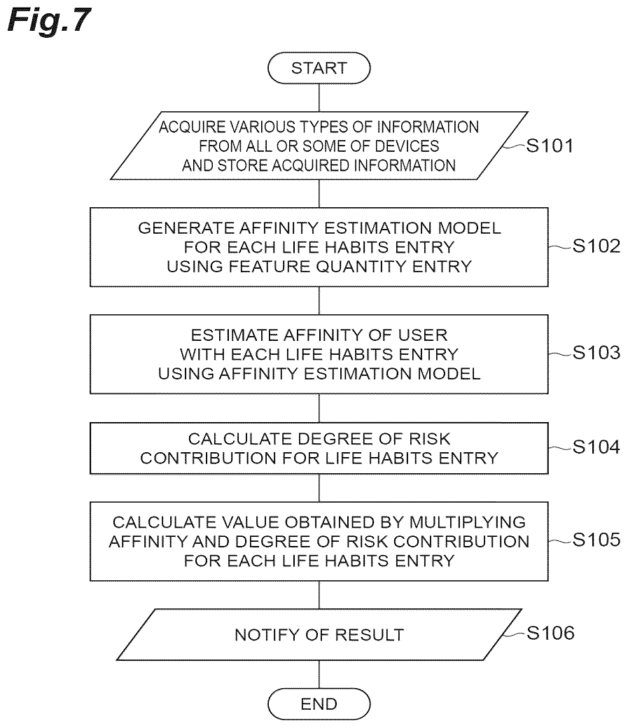

FIG. 7 is a flowchart illustrating operations of the user behavior proposal device 100 and the data collection and analysis device 200 according to the present disclosure.

In the data collection and analysis device 200, the data acquiring unit 201 acquires terminal log information from all or some of the devices such as the attribute information acquisition device 30, and the data storage unit 202 stores the terminal log information for training. The data acquiring unit 201 acquires improvement data indicating whether a life habits entry (a user behavior) has been improved on the basis of a behavioral change message for each life habits entry, and the data storage unit 202 stores the improvement data (S101).

The affinity estimation model generating unit 203 generates an affinity estimation model 100a for each life habits entry (such as a step count and hours of sleep) using the acquired terminal log information and the acquired improvement data of each life habits entry (S102). In the present disclosure, a plurality of affinity estimation models 100a are generated, transmitted to the user behavior proposal device 100, and registered therein.

In the user behavior proposal device 100, a smartphone log 301 of a user (a terminal 300) to be estimated is received, and the affinity calculating unit 102 calculates an affinity for each life habits entry using the smartphone log 301 and the affinity estimation model 100a (S103). The risk contribution degree calculating unit 103 calculates a degree of risk contribution for each life habits entry (S104).

The affinity and risk contribution degree calculating unit 104 calculates an affinity and a degree of risk contribution for each life habits entry (S105).

The information transmitting unit 105 transmits the calculated affinity and the calculated degree of risk contribution as a result to the terminal 300 (S106). In the result, it is preferable that the life habits entries be arranged in the descending order of values obtained by multiplying the affinity and the degree of risk contribution. Accordingly, it is possible to easily notify a user of a recommended behavior.

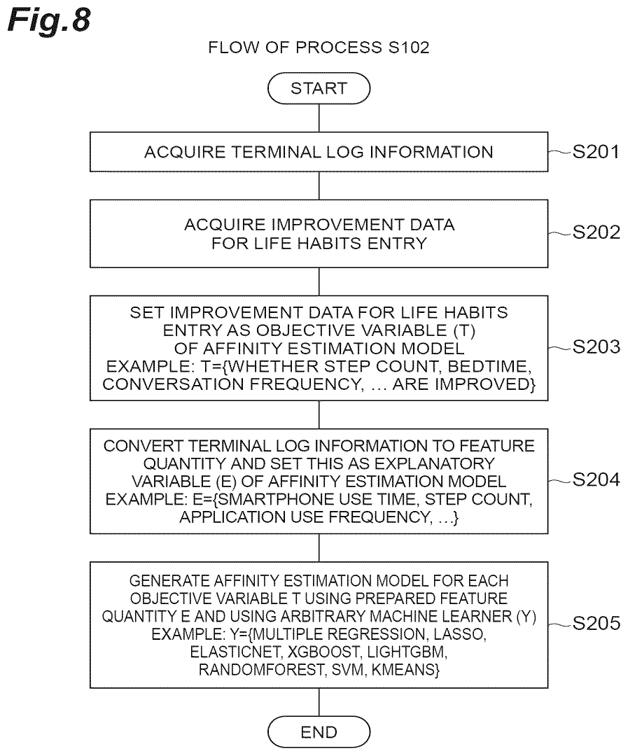

Details of the process of generating an affinity estimation model 100a in S102 will be described below. FIG. 8 is a flowchart illustrating operations of the data collection and analysis device 200.

The data acquiring unit 201 acquires the terminal log information from various acquisition devices such as the attribute information acquisition device 30 (S201). The data acquiring unit 201 acquires improvement data for each life habits entry on the basis of the terminal log information (S202). The data storage unit 202 stores types of behavioral change messages (such as a behavioral change target) and a date and time of transmission in advance, and the data acquiring unit 201 determines whether the corresponding life habits entry has been improved on the basis of the terminal log information based on the behavioral change target (life habits entry) and the date and time of transmission. The data acquiring unit 201 acquires improvement data for each life habits entry on the basis of the determination result. This improvement data is acquired for each user and for each life habits entry.

The affinity estimation model generating unit 203 uses the improvement data for each life habits entry as an objective variable (T) of the affinity estimation model. For example, T={step count, wake-up time, conversation frequency, . . . } is set (S203).

The affinity estimation model generating unit 203 converts the terminal log information to feature quantities and uses the feature quantities as explanatory variables (E) of an affinity estimation model (S204). For example, E={smartphone use time, step count, application use frequency, . . .} is set.

Then, the affinity estimation model generating unit 203 generates an affinity estimation model 100a for each objective variable T using the prepared feature quantities E as explanatory variables and using an arbitrary machine learner (Y) (S205). Examples of the arbitrary machine learner include Y={multiple regression, Lasso, ElasticNet, XGBoost, LightGBM, RandomForest, SVM, Kmeans}

In this way, it is possible to generate a plurality of affinity estimation models 100a for each life habits entry.

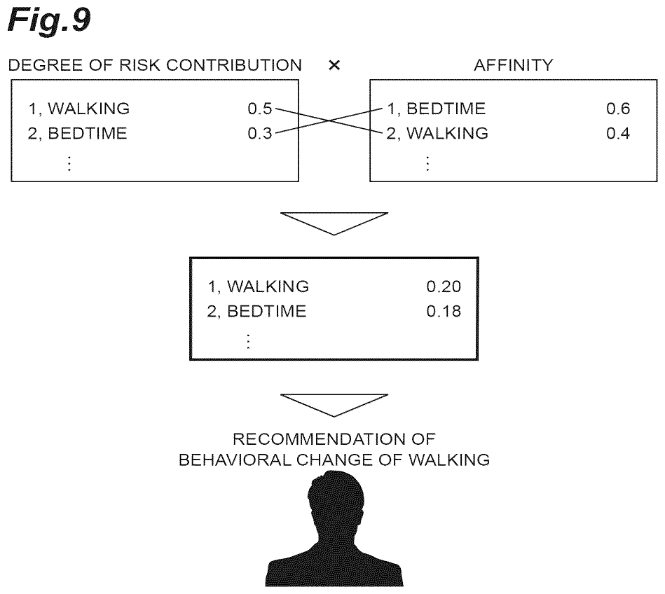

The process of S106 will be described below. As described above, the result is generated on the basis of a value obtained by multiplying the degree of risk contribution and the affinity. FIG. 9 is a diagram schematically illustrating the result. As illustrated in the drawing, the value obtained by multiplying the degree of risk contribution and the affinity indicates a degree of recommendation of a behavioral change. When the user behavior proposal device 100 notifies a user of the degree of recommendation of a behavioral change, the user can ascertain the degree of recommendation of the corresponding life habits entry.

In FIG. 9, when the degree of risk contribution of walking of a user is 0.5 and the affinity of walking is 0.4, the degree of recommendation of walking of the user is 0.20. This degree of recommendation of a behavioral change indicates a behavior with a degree of risk contribution to health and an affinity with the user (acceptable), and the higher degree of recommendation of a behavioral change means a life habits entry which is more executable by the user and which has higher efficiency in view of promotion of health.

Accordingly, when a life habits entry has a high affinity and is a behavior with low efficiency in promotion of health, the value decreases and the user can ascertain that it is not necessary to select the behavior. On the other hand, when a life habits entry is a behavior with high efficiency in promotion of health and has a low affinity, the user has difficulty accepting it.

Priority may be given to the affinity, and a life habits entry of which the affinity is equal to or greater than a predetermined value may be notified of. Since a user has difficulty accepting a life habits entry with a low affinity, there is a high likelihood that proposal of such a life habits entry will not cause improvement.

Operations and advantages of the user behavior proposal device 100 according to the present disclosure will be described below. In the user behavior proposal device 100 according to the present disclosure, the affinity calculating unit 102 calculates an affinity of one user with each life habits entry on the basis of a terminal log of the one user. The information transmitting unit 105 transmits the calculated affinity along with a behavior thereof to the one user. The information transmitting unit 105 may notify the one user of a behavior based on an affinity satisfying a predetermined as a recommended behavior as information based on the affinity.

With this configuration, it is possible to notify of information based on an affinity of a user with a life habits entry (a behavior) and to promote life improvement of the user on the basis of the information. When an affinity itself is received, a user can easily determine what behavior of a life habits entry to adopt. A user having received a life habits entry with an affinity can easily adopt the behavior of the life habits entry. Accordingly, it is possible to contribute to improvement of a life habits entry by determining the life habits entry with an affinity and notifying of the life habits entry.

In the user behavior proposal device 100 according to the present disclosure, the risk contribution degree calculating unit 103 calculates a degree of risk contribution of a user's behavior on the basis of a smartphone log 301 of the user (terminal 300). The affinity and risk contribution degree calculating unit 104 calculates a degree of recommendation of a behavioral change of the life habits entry of the user on the basis of the degree of risk contribution and the affinity. The information transmitting unit 105 transmits the degree of recommendation of a behavioral change to the user (the terminal 300). The affinity and risk contribution degree calculating unit 104 and the information transmitting unit 105 have a function of deriving a recommended behavior of the user on the basis of the degree of recommendation of a behavioral change.

With this configuration, the user having received the degree of recommendation of a behavioral change can easily determine what life habits entry the user has to adopt on the basis of the degree of risk contribution in a user behavior (the degree of risk contribution of the user behavior for health) and the affinity (acceptability of the user behavior). In the present disclosure, the user is notified of a score obtained by multiplying the affinity and the degree of risk contribution, but may be notified of only the affinity.

In the user behavior proposal device 100 according to the present disclosure, the risk contribution degree calculating unit 103 calculates the degree of risk contribution for each life habits entry which is one of a plurality of predetermined behaviors. The affinity and risk contribution degree calculating unit 104 calculates the degree of recommendation of a behavioral change based on the affinity and the degree of risk contribution for each life habits entry. The information transmitting unit 105 transmits the degree of recommendation of a behavioral change for each life habits entry which is a predetermined behavior to the terminal 300. That is, the affinity and risk contribution degree calculating unit 104 and the information transmitting unit 105 serve as a recommended behavior deriving unit and derive a life habits entry of a recommended behavior using the affinity and the degree of risk contribution.

In the user behavior proposal device 100 according to the present disclosure, the affinity calculating unit 102 determines a life habits entry with an affinity with a user by calculating the affinity for each life habits entry of the user using an affinity estimation model 100a. The affinity estimation model 100a is generated on the basis of a positive user who has adopted a behavior based on a behavioral change message corresponding to a predetermined suggestion for a predetermined behavior (a life habits entry) and a negative user who has not adopted a behavior based on the behavioral change message. The affinity estimation model 100a is an estimation model for estimating an affinity with a life habits entry.

With this configuration, it is possible to accurately estimate an affinity of a user with a life habits entry using the affinity estimation model 100a for estimating an affinity with the life habits entry.

The affinity estimation model 100a is generated using terminal log information of a terminal 300a, attribute information, and a questionnaire answer result as explanatory variables and using information indicating whether a user has conformed to a behavioral change message which is a suggestion as an objective variable.

Regarding the affinity estimation model 100a, a plurality of users are notified of a behavioral change message including a target value of a life habits entry as a suggestion, a positive user or a negative user is determined with respect to the notification timing, and the affinity estimation model 100a is generated on the basis of the determination result.

The target value is determined on the basis of a value obtained by performing a statistical process on the life habits entries of the plurality of users.

The positive user and the negative user are determined as follows. That is, a user is determined to be a positive user having adopted a behavior corresponding to a suggestion when a predetermined behavior of the user indicated by one log can be determined to correspond to the suggestion better in comparison with the predetermined behavior of the user in the past on the basis of terminal logs stored in a log storage unit, and a user is determined to be negative user not having adopted the behavior corresponding to the suggestion when the predetermined behavior can be determined to correspond to the suggestion worse in comparison with the behavior of the user in the past.

In the present disclosure, a message for prompting a predetermined behavior of a plurality of users associated with one log is transmitted to the plurality of users, a positive user and a negative user are determined by determining whether a predetermined behavior has been adopted before and after the message is transmitted, and an affinity estimation model 100a is generated on the basis of the determination result.

In the present disclosure, the affinity estimation model 100a is generated by the data collection and analysis device 200. That is, in the data collection and analysis device 200, the data acquiring unit 201 acquires terminal log information of each user. The data acquiring unit 201 determines whether a user is a positive user or a negative user from the terminal log information and a transmission date and time of a behavioral change message and acquires improvement data.

Then, the data storage unit 202 stores the terminal log information and the improvement data of each user.

Then, in acquiring the improvement data, the data acquiring unit 201 determines that the user is a positive user having adopted a behavior corresponding to a suggestion indicated by the behavioral change message when a life habits entry (a predetermined behavior) of the user indicated by one log can be determined to correspond to the suggestion of the behavioral change message better in comparison with the predetermined behavior in the past on the basis of the terminal log information of each of the plurality of users. On the other hand, the data acquiring unit 201 determines that the user is a negative user having not adopted the behavior corresponding to the suggestion indicated by the behavioral change message when the life habits entry can be determined to correspond to the suggestion of the behavioral change message worse in comparison with the life habits entry of the user in the past. Accordingly, the data acquiring unit 201 can determine a positive user and a negative user and acquire the improvement data.

That is, the data acquiring unit 201 determines a positive user and a negative user by determining whether a life habits entry has been improved before and after the behavioral change message for prompting a predetermined behavior of a user associated with one piece of terminal log information has been transmitted for a plurality of users.

Through this process, it is possible to determine whether a user conforms to a behavioral change message, that is, to generate an affinity estimation model for estimating whether the user can easily adopt the life habits entry. It is possible to easily improve the life habits entry of the user using the affinity estimation model and using a terminal log such as a smartphone log.

The data collection and analysis device 200 serving as an estimation model generation device according to the present disclosure includes the data storage unit 202 configured to store training terminal logs of a plurality of users, the data acquiring unit 201 configured to acquire a positive user having adopted a behavior corresponding to a predetermined suggestion (a behavioral change message) for a behavior of the user and a negative user not having adopted the behavior corresponding to the predetermined suggestion out of the plurality of users on the basis of the training terminal logs, and the affinity estimation model generating unit 203 configured to generate an estimation model for estimating an affinity with a plurality of behaviors (life habits entries) on the basis of the training terminal log, the positive user, and the negative user.

The user behavior suggestion device 100 of the present disclosure comprises the following components.
[1]

A user behavior proposal device comprising:
an affinity calculating unit configured to calculate an affinity with each of a plurality of behaviors of one user on the basis of at least one of a terminal log of the one user, user attributes, and a questionnaire result; and
an output unit configured to output a behavior with a high affinity as a recommended behavior to the one user.
[2]

The user behavior proposal device according to [1], wherein the output unit notifies the one user of the behavior with a high affinity and the affinity of the behavior as the recommended behavior.
[3]

The user behavior proposal device according to [1] or [2] claim 1, further comprising:
a risk contribution degree calculating unit configured to calculate a degree of risk contribution of each of the plurality of behaviors of the user on the basis of the terminal log of the user; and
a recommended behavior deriving unit configured to derive a recommended behavior of the user on the basis of the degree of risk contribution and the affinity,
wherein the output unit outputs the recommended behavior to the user.
[4]

The user behavior proposal device according to [3], wherein the affinity calculating unit calculates the affinity for each of the plurality of behaviors,
wherein the risk contribution degree calculating unit calculates the degree of risk contribution for each of the plurality of behaviors, and
wherein the recommended behavior deriving unit derives the recommended behavior using the affinity and the degree of risk contribution.
[4]

The user behavior proposal device according to any one of [1] to [4], wherein the affinity calculating unit calculates the affinity for each of the plurality of behaviors of the user on the basis of an estimation model for estimating the affinity of the plurality of behaviors which is generated on the basis of a positive user who has adopted a behavior corresponding to a predetermined suggestion for the plurality of behaviors and a negative user who has not adopted the behavior corresponding to the predetermined suggestion.
[6]

The user behavior proposal device according to [5], wherein the estimation model is generated using at least one of training terminal logs of users having received the predetermined suggestion, user attributes, and questionnaire answer results of the users as explanatory variables and using information indicating whether the predetermined suggestion has been employed as an objective variable.
[7]

The user behavior proposal device according to [5] or [6], wherein a plurality of users are notified of a message including target values of the plurality of behaviors as the suggestion, and a positive user or a negative user is determined at the time of notification, and
wherein the estimation model is generated on the basis of the determination result.
[8]

The user behavior proposal device according to [7], wherein the target values are determined on the basis of a value obtained by statistically processing behaviors of each of the plurality of users.
[9]

The user behavior proposal device according to any one of [5] to [8], wherein a positive user who has adopted a behavior corresponding to the suggestion for a user's behavior is determined when the user's behavior is determined to correspond to the suggestion in comparison with the user's past behavior, and a negative user who has not adopted the behavior corresponding to the suggestion for the behavior is determined when the user's behavior is determined not to correspond to the suggestion in comparison with the user's past behavior.
[10]

An estimation model generation device comprising:
a storage unit configured to store training information including at least one of training terminal logs of a plurality of users, user attributes, and questionnaire results;
an acquisition unit configured to acquire a positive user who has adopted a behavior corresponding to a predetermined suggestion for a user's behavior and a negative user who has not adopted the behavior corresponding to the predetermined suggestion for each of the plurality of users on the basis of the training information; and
an affinity estimation model generating unit configured to generate an estimation model for estimating an affinity with the behavior on the basis of the training information, the positive user, and the negative user.

The block diagram used for the description of the above embodiments shows blocks of functions. Those functional blocks (component parts) are implemented by any combination of at least one of hardware and software. Further, a means of implementing each functional block is not particularly limited. Specifically, each functional block may be implemented by one physically or logically combined device or may be implemented by two or more physically or logically separated devices that are directly or indirectly connected (e.g., by using wired or wireless connection etc.). The functional blocks may be implemented by combining software with the above-described one device or the above-described plurality of devices.

The functions include determining, deciding, judging, calculating, computing, processing, deriving, investigating, looking up/searching/inquiring, ascertaining, receiving, transmitting, outputting, accessing, resolving, selecting, choosing, establishing, comparing, assuming, expecting, considering, broadcasting, notifying, communicating, forwarding, configuring, reconfiguring, allocating/mapping, assigning and the like, though not limited thereto.

For example, the functional block (component part) that implements the function of transmitting is referred to as a transmitting unit or a transmitter. In any case, a means of implementation is not particularly limited as described above.

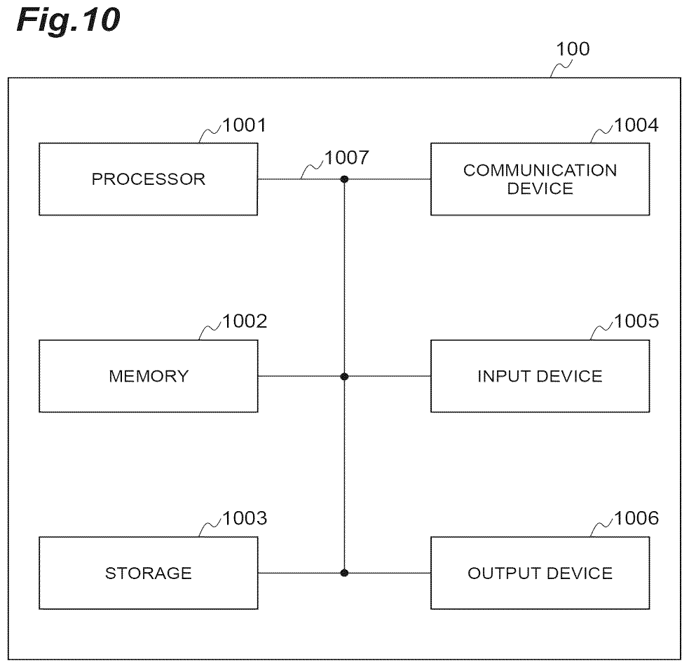

For example, the user behavior proposal device 100 and the data collection and analysis device 200 according to one embodiment of the present disclosure may function as a computer that performs processing of a user behavior proposal method according to the present disclosure. FIG. 10 is a view showing an example of the hardware configuration of the user behavior proposal device 100 and the data collection and analysis device 200 according to one embodiment of the present disclosure. The user behavior proposal device 100 and the data collection and analysis device 200 described above may be physically configured as a computer device that includes a processor 1001, a memory 1002, a storage 1003, a communication device 1004, an input device 1005, an output device 1006, a bus 1007 and the like.

In the following description, the term “device” may be replaced with a circuit, a device, a unit, or the like. The hardware configuration of the user behavior proposal device 100 and the data collection and analysis device 200 may be configured to include one or a plurality of the devices shown in the drawings or may be configured without including some of those devices.

The functions of the user behavior proposal device 100 and the data collection and analysis device 200 may be implemented by loading predetermined software (programs) on hardware such as the processor 1001 and the memory 1002, so that the processor 1001 performs computations to control communications by the communication device 1004 and control at least one of reading and writing of data in the memory 1002 and the storage 1003.

The processor 1001 may, for example, operate an operating system to control the entire computer. The processor 1001 may be configured to include a CPU (Central Processing Unit) including an interface with a peripheral device, a control device, an arithmetic device, a register and the like. For example, the affinity calculating unit 102, a risk contribution degree calculating unit, and The affinity and risk contribution degree calculating unit 104 and the like described above may be implemented by the processor 1001.

Further, the processor 1001 loads a program (program code), a software module and data from at least one of the storage 1003 and the communication device 1004 into the memory 1002 and performs various processing according to them. As the program, a program that causes a computer to execute at least some of the operations described in the above embodiments is used. For example, the affinity calculating unit 102 may be implemented by a control program that is stored in the memory 1002 and operates on the processor 1001, and the other functional blocks may be implemented in the same way. Although the above-described processing is executed by one processor 1001 in the above description, the processing may be executed simultaneously or sequentially by two or more processors 1001. The processor 1001 may be implemented in one or more chips. Note that the program may be transmitted from a network through a telecommunications line.

The memory 1002 is a computer-readable recording medium, and it may be composed of at least one of ROM (Read Only Memory), EPROM (ErasableProgrammable ROM), EEPROM (Electrically ErasableProgrammable ROM), RAM (Random Access Memory) and the like, for example. The memory 1002 may be also called a register, a cache, a main memory (main storage device) or the like. The memory 1002 can store a program (program code), a software module and the like that can be executed for implementing the user behavior proposal method according to one embodiment of the present disclosure.

The storage 1003 is a computer-readable recording medium, and it may be composed of at least one of an optical disk such as a CD-ROM (Compact Disk ROM), a hard disk drive, a flexible disk, a magneto-optical disk (e.g., a compact disk, a digital versatile disk, and a Blu-ray (registered trademark) disk), a smart card, a flash memory (e.g., a card, a stick, and a key drive), a floppy (registered trademark) disk, a magnetic strip and the like, for example. The storage 1003 may be called an auxiliary storage device. The above-described storage medium may be a database, a server, or another appropriate medium including at least one of the memory 1002 and/or the storage 1003, for example.

The communication device 1004 is hardware (a transmitting and receiving device) for performing communication between computers via at least one of a wired network and a wireless network, and it may also be referred to as a network device, a network controller, a network card, a communication module, or the like. The communication device 1004 may include a high-frequency switch, a duplexer, a filter, a frequency synthesizer or the like in order to implement at least one of FDD (Frequency Division Duplex) and TDD (Time Division Duplex), for example. For example, the above-described information input unit 101 and the information transmitting unit 105 or the like may be implemented by the communication device 1004. The communication device 1004 may be implemented in such a way that a transmitting unit and a receiving unit are physically or logically separated.

The input device 1005 is an input device (e.g., a keyboard, a mouse, a microphone, a switch, a button, a sensor, etc.) that receives an input from the outside. The output device 1006 is an output device (e.g., a display, a speaker, an LED lamp, etc.) that makes output to the outside. Note that the input device 1005 and the output device 1006 may be integrated (e.g., a touch panel).

In addition, the devices such as the processor 1001 and the memory 1002 are connected by the bus 1007 for communicating information. The bus 1007 may be a single bus or may be composed of different buses between different devices.

Further, the user behavior proposal device 100 may include hardware such as a microprocessor, a DSP (Digital Signal Processor), an ASIC (Application Specific Integrated Circuit), a PLD (Programmable Logic Device), and an FPGA (Field Programmable Gate Array), and some or all of the functional blocks may be implemented by the above-described hardware components. For example, the processor 1001 may be implemented with at least one of these hardware components.

Notification of information may be made by another method, not limited to the aspects/embodiments described in the present disclosure. For example, notification of information may be made by physical layer signaling (e.g., DCI (Downlink Control Information), UCI (Uplink Control Information)), upper layer signaling (e.g. RRC (Radio Resource Control) signaling, MAC (Medium Access Control) signaling, annunciation information (MIB (Master Information Block), SIB (System Information Block))), another signal, or a combination of them. Further, RRC signaling may be called an RRC message, and it may be an RRC Connection Setup message, an RRC Connection Reconfiguration message or the like, for example.

The procedure, the sequence, the flowchart and the like in each of the aspects/embodiments described in the present disclosure may be in a different order unless inconsistency arises. For example, for the method described in the present disclosure, elements of various steps are described in an exemplified order, and it is not limited to the specific order described above.

Input/output information or the like may be stored in a specific location (e.g., memory) or managed in a management table. Further, input/output information or the like can be overwritten or updated, or additional data can be written. Output information or the like may be deleted. Input information or the like may be transmitted to another device.

The determination may be made by a value represented by one bit (0 or 1), by a truth-value (Boolean: true or false), or by numerical comparison (e.g., comparison with a specified value).

Each of the aspects/embodiments described in the present disclosure may be used alone, may be used in combination, or may be used by being switched according to the execution. Further, a notification of specified information (e.g., a notification of “being X”) is not limited to be made explicitly, and it may be made implicitly (e.g., a notification of the specified information is not made).

Although the present disclosure is described in detail above, it is apparent to those skilled in the art that the present disclosure is not restricted to the embodiments described in this disclosure. The present disclosure can be implemented as a modified and changed form without deviating from the spirit and scope of the present disclosure defined by the appended claims. Accordingly, the description of the present disclosure is given merely by way of illustration and does not have any restrictive meaning to the present disclosure.

Software may be called any of software, firmware, middle ware, microcode, hardware description language or another name, and it should be interpreted widely so as to mean an instruction, an instruction set, a code, a code segment, a program code, a program, a sub-program, a software module, an application, a software application, a software package, a routine, a sub-routine, an object, an executable file, a thread of execution, a procedure, a function and the like.

Further, software, instructions and the like may be transmitted and received via a transmission medium. For example, when software is transmitted from a website, a server or another remote source using at least one of wired technology (a coaxial cable, an optical fiber cable, a twisted pair and a digital subscriber line (DSL) etc.) and wireless technology (infrared rays, microwave etc.), at least one of those wired technology and wireless technology are included in the definition of the transmission medium.

The information, signals and the like described in the present disclosure may be represented by any of various different technologies. For example, data, an instruction, a command, information, a signal, a bit, a symbol, a chip and the like that can be referred to in the above description may be represented by a voltage, a current, an electromagnetic wave, a magnetic field or a magnetic particle, an optical field or a photon, or an arbitrary combination of them.

Note that the term described in the present disclosure and the term needed to understand the present disclosure may be replaced by a term having the same or similar meaning. For example, at least one of a channel and a symbol may be a signal (signaling). Further, a signal may be a message. Furthermore, a component carrier (CC) may be called a cell, a frequency carrier, or the like.

Further, information, parameters and the like described in the present disclosure may be represented by an absolute value, a relative value to a specified value, or corresponding different information. For example, radio resources may be indicated by an index.

The names used for the above-described parameters are not definitive in any way. Further, mathematical expressions and the like using those parameters are different from those explicitly disclosed in the present disclosure in some cases. Because various channels (e.g., PUCCH, PDCCH etc.) and information elements (e.g., TPC etc.) can be identified by every appropriate names, various names assigned to such various channels and information elements are not definitive in any way.

In the present disclosure, the terms such as “Mobile Station (MS)” “user terminal”, “User Equipment (UE)” and “terminal” can be used to be compatible with each other.

The mobile station can be also called, by those skilled in the art, a subscriber station, a mobile unit, a subscriber unit, a wireless unit, a remote unit, a mobile device, a wireless device, a wirelesscommunication device, a remote device, a mobile subscriber station, an access terminal, a mobile terminal, a wireless terminal, a remote terminal, a handset, a user agent, a mobile client, a client or several other appropriate terms.

Note that the term “determining” and “determining” used in the present disclosure includes a variety of operations. For example, “determining” and “determining” can include regarding the act of judging, calculating, computing, processing, deriving, investigating, looking up/searching/inquiring (e.g., looking up in a table, a database or another data structure), ascertaining or the like as being “determined” and “determined”. Further, “determining” and “determining” can include regarding the act of receiving (e.g., receiving information), transmitting (e.g., transmitting information), inputting, outputting, accessing (e.g., accessing data in a memory) or the like as being “determined” and “determined”. Further, “determining” and “determining” can include regarding the act of resolving, selecting, choosing, establishing, comparing or the like as being “determined” and “determined”. In other words, “determining” and “determining” can include regarding a certain operation as being “determined” and “determined”. Further, “determining (determining)” may be replaced with “assuming”, “expecting”, “considering”and the like.

The term “connected”, “coupled” or every transformation of this term means every direct or indirect connection or coupling between two or more elements, and it includes the case where there are one or more intermediate elements between two elements that are “connected” or “coupled” to each other. The coupling or connection between elements may be physical, logical, or a combination of them. For example, “connect” may be replaced with “access”. When used in the present disclosure, it is considered that two elements are “connected” or “coupled” to each other by using at least one of one or more electric wires, cables, and printed electric connections and, as several non-definitive and non-comprehensive examples, by using electromagnetic energy such as electromagnetic energy having a wavelength of a radio frequency region, a microwave region and an optical (both visible and invisible) region.

The description “on the basis of” used in the present disclosure does not mean “only on the basis of” unless otherwise noted. In other words, the description “on the basis of” means both of “only on the basis of”and “at least on the basis of”.

When the terms such as “first” and “second” are used in the present disclosure, any reference to the element does not limit the amount or order of the elements in general. Those terms can be used in the present disclosure as a convenient way to distinguish between two or more elements. Thus, reference to the first and second elements does not mean that only two elements can be adopted or the first element needs to precede the second element in a certain form.

As long as “include”, “including” and transformation of them are used in the present disclosure, those terms are intended to be comprehensive like the term “comprising”. Further, the term “or” used in the present disclosure is intended not to be exclusive OR.

In the present disclosure, when articles, such as “a”, “an”, and “the” in English, for example, are added by translation, the present disclosure may include that nouns following such articles are plural.

In the present disclosure, the term “A and B are different” may mean that “A and B are different from each other”. Note that this term may mean that “A and B are different from C”. The terms such as “separated” and “coupled” may be also interpreted in the same manner.

### REFERENCE SIGNS LIST

20 . . . Terminal use log collection device, 30 . . . Attribute information acquisition device, 40 . . . Position information acquisition device, 50 . . . Terminal operation information acquisition device, 60 . . . Application use information acquisition device, 70 . . . Healthcare information acquisition device, 100 . . . User behavior proposal device, 100a . . . Affinity estimation model, 101 . . . Information input unit, 102 . . . Affinity calculating unit, 103 . . . Risk contribution degree calculating unit, 104 . . . Affinity risk contribution degree calculating unit, 105 . . . Information transmitting unit, 200 . . . Data collection and analysis device, 201 . . . Data acquiring unit, 202 . . . Data storage unit, 203 . . . Affinity estimation model generating unit, 204 . . . Transmission unit, 300, 300a . . . Terminal, 301 . . . Smartphone log

1. A user behavior proposal device comprising:
an affinity calculating unit configured to calculate an affinity with each of a plurality of behaviors of one user on the basis of at least one of a terminal log of the one user, user attributes, and a questionnaire result; and
an output unit configured to output a behavior with a high affinity as a recommended behavior to the one user.

2. The user behavior proposal device according to claim 1, wherein the output unit notifies the one user of the behavior with a high affinity and the affinity of the behavior as the recommended behavior.

3. The user behavior proposal device according to claim 1, further comprising:
a risk contribution degree calculating unit configured to calculate a degree of risk contribution of each of the plurality of behaviors of the user on the basis of the terminal log of the user; and
a recommended behavior deriving unit configured to derive a recommended behavior of the user on the basis of the degree of risk contribution and the affinity,
wherein the output unit outputs the recommended behavior to the user.

4. The user behavior proposal device according to claim 3, wherein the affinity calculating unit calculates the affinity for each of the plurality of behaviors,
wherein the risk contribution degree calculating unit calculates the degree of risk contribution for each of the plurality of behaviors, and
wherein the recommended behavior deriving unit derives the recommended behavior using the affinity and the degree of risk contribution.

5. The user behavior proposal device according to claim 1, wherein the affinity calculating unit calculates the affinity for each of the plurality of behaviors of the user on the basis of an estimation model for estimating the affinity of the plurality of behaviors which is generated on the basis of a positive user who has adopted a behavior corresponding to a predetermined suggestion for the plurality of behaviors and a negative user who has not adopted the behavior corresponding to the predetermined suggestion.

6. The user behavior proposal device according to claim 5, wherein the estimation model is generated using at least one of training terminal logs of users having received the predetermined suggestion, user attributes, and questionnaire answer results of the users as explanatory variables and using information indicating whether the predetermined suggestion has been employed as an objective variable.

7. The user behavior proposal device according to claim 5, wherein a plurality of users are notified of a message including target values of the plurality of behaviors as the suggestion, and a positive user or a negative user is determined at the time of notification, and
wherein the estimation model is generated on the basis of the determination result.

8. The user behavior proposal device according to claim 7, wherein the target values are determined on the basis of a value obtained by statistically processing behaviors of each of the plurality of users.

9. The user behavior proposal device according to claim 5, wherein a positive user who has adopted a behavior corresponding to the suggestion for a user's behavior is determined when the user's behavior is determined to correspond to the suggestion in comparison with the user's past behavior, and a negative user who has not adopted the behavior corresponding to the suggestion for the behavior is determined when the user's behavior is determined not to correspond to the suggestion in comparison with the user's past behavior.

10. An estimation model generation device comprising:
a storage unit configured to store training information including at least one of training terminal logs of a plurality of users, user attributes, and questionnaire results;
an acquisition unit configured to acquire a positive user who has adopted a behavior corresponding to a predetermined suggestion for a user's behavior and a negative user who has not adopted the behavior corresponding to the predetermined suggestion for each of the plurality of users on the basis of the training information; and
an affinity estimation model generating unit configured to generate an estimation model for estimating an affinity with the behavior on the basis of the training information, the positive user, and the negative user.
